(19) United States
(12) Patent Application Publication
(10) Pub. No.: US 2011/0009713 A1
(43) Pub. Date: Jan. 13, 2011

(54) Rhythmic percussion exercise garment with electronic interface and method of conducting an exercise program

(76) Inventor: Nomi Feinberg, Lakewood, NJ (US)

Correspondence Address: Law Offices Eric R. Benson, Esq., 6A Hillside Lane, Westford, VT, 05494-9769, US

(21) Appl. No.: 12/806,765

(22) Filed: Aug. 20, 2010

Related U.S. Application Data
(63) Continuation-in-part of application No. 12/321,508, filed on Jan. 22, 2009.

(51) Int. Cl. A61B 5/00; A63B 71/00

(52) U.S. Cl. 600/301; 482/105; 482/8

(57) ABSTRACT

Hands-free exercise device combining: weights; an electronic vital signs monitoring device with interactive capabilities and a controllable interface with an electronic or instrumental percussive music source, adapted to be worn about the user's body resulting in new method of conducting an exercise program. User varies weight to adjust muscle load during a range of motion. Simultaneously, sound is movement activated: depending on placement, user learns to exercise de-conditioned muscle groups by learning how to produce sound. During exercise, device monitors/records user's vital signs to objectively determine exertion level in real time, and calculate long term progress. Device can automatically adjust tempo, challenging user to respond with concordant change in exercise pace so that actual exertion level will increase or decrease to desired levels. Device can respond to exertion, notifying user when a threshold of exertion is reached by switching the type of instrumental sound, providing indicative audible cue.

### CROSS REFERENCE TO RELATED APPLICATION

This application is a Continuation-In-Part of the Co-Pending patent application U.S. Continuation-in Part patent application, U.S. Ser. No. 12/321,508, which was filed on Jan. 22, 2009.

### FIELD OF THE INVENTION

The present invention generally relates to the combination of musical instruments, a user monitoring and rhythmic controlling and “vital signs” monitoring electronic device and worn exercise equipment, weighted for the purpose of adding or removing a muscle load. In particular, the present invention relates to “percussive type” instruments, such as agogo, agung/agong, agung a tamlang, ahoko, array mbira, babendil, bell, bianqing, bianzhong, bones, boomwhackers, cajón, carillon, castanets, caxixi, chime, ching, clapper, claves, cowbell, crotales, cymbal, zil (finger cymbal), cymbalum, an electronic movement actuated means of producing music, fangxiang, gamelan, american gamelan, gangsa, gendér, ugal, gandingan, gandingan a kayo, ganzá, ghatam, glass marimba, glass harmonica, glockenspiel, gong, guban, guiro, handbells, hang, hosho, ipu, kagul, kalimba, kulintang/kolintang, kulintang a kayo, kulintang a tiniok, lamellaphone, luntang, maracas, marimba, marimbaphone, mbira, metallophone, musical saw, rainstick, ranat ek lek, ranat thum lek, ratchet, rattle, riq, shekere, singing bowl, slit drum, spoons, steelpan, tambourine, triangle, trychel, timpani, tubular bells, udu, vibraphone, vibraslap, washboard, whip, xylophone, xylorimba, and free weights placed side by side in a hands free exercise garment, and more particularly to a band construction which assists in confining the shaker and weights in the said band to allow for variation in both the type of shaker sound and the amount of added weight, so that as a user exercises, the user simultaneously receives the benefits of sustained weight training while the user actually “plays” the percussive instrument through body movements which activate the shaker adding a desired rhythmic accompaniment. At the same time an electronic monitoring device monitors the user's exercise expenditure, percentage of maximal heart rate and VO2 max by monitoring the user's life signs, such as their pulse, resting heart rate, blood pressure, saturated blood oxygen level, body temperature, respiratory rate, and VO2 max whereby the user may monitor the same and the device is thereby interactive with the exercise music source: according to the program mode chosen by the user, the user's fitness instructor or the user's physician the device may “lead” the user by automatically adjusting the speed of the music and/or type of musical sound produced by the device such that the “beat” of the music and/or the percussive sound, when followed by the user, will produce preset exercise expenditure by one or more desired parameters based upon the life signs being monitored, pre-settings may be automatically consistent to objective standards recommended by the health and fitness industry which, for example, formulate the appropriate percentage of maximal heart rate according to age, weight and resting heart rate, whereby the user's personal data have been entered into the preset formula; or exercise expenditure thresholds can be subjectively set by the user according to personal training goals. The electronic device can also be programmed to “follow” the user, such that at the moment the user reaches preset thresholds of energy expenditure, based on the vital signs being monitored, set either by industry standard, an instructor working with the user, either alone or in a class setting, or by the user's personal exercise goals, the device will produce a distinctly different beat or different musical instrument sound, which thereby audibly indicates to the user in real time that he has transitioned from one phase of exercise, such as the warm up, into another phase of the workout, such as sustained aerobic training zone, cool down, or another phase which may be undesirable such as beyond the recommended percentage of maximal heart rate, whereby a warning signal will be emitted. The device of the present invention will allow the user to keep track over time his progress, such as how long it takes to attain different phases of the workout, the length of time shortening as the user moves from de-conditioned to conditioned. The electronic device can also be programmed for different types of exercise regimens to signal to the user the different levels of the program and if desired automatically adjust the speed of the music or the type of musical sound produced by the device to which the user has set their exercise pace. Traditionally, aerobic exercise routines consist of three phases: starting with a warm-up prior to strenuous activity, which is meant to stretch and loosens muscles and signal to the brain that demands for system wide oxygen are slowly increasing, in turn placing greater work on the heart to supply oxygen rich blood; subsequent to the warm-up aerobic exercise enters a sustained strenuous movements or training phase during which, according to one's goals, exercise expenditure may be consistent or intermittent, but kept within healthful parameters of vital sign indicators such as heart rate, blood pressure, and percentage of maximal heart rate; and finally aerobic exercise recedes into a cool down phase, whereby the demands on the body for greater exercise are slowly reduced back to the non-exercising level. In all program modes, leading or following the user, the device of the present invention is used as an audible indicator in real time and with both changes in tempo and in changes in sound, that an exercise expenditure level has been attained or the user must strive to attain it: for example, as long as the exercise device is playing a marraca sound, the user knows that he is in the warm-up stage. If the device is set to follow the user activated movement, when the exercise device changes from a marraca to a bell sound, this change indicates to the user that energy expenditure has reached the strenuous stage. Then, for example, when the sound changes again to a shekere, the user knows he has been in the training zone for a certain amount of time, set as desired for example 20, 30 or 40 minutes. Or if the said device is set to inspire user activated movement, then a change in instrumental sound or tempo, indicates that the user must respond to the device's lead and work more strenuously; or has reached the desired energy expenditure level. This setting works well for interval training whereby the device is set in intervals, so that the tempo and/or instrument sound changes during the strenuous phase to indicate to the user WHEN to adjust energy expenditure, and HOW to attain the desired energy expenditure by responding to the new given, tempo which is set to reflect interval time periods of rest versus more strenuous bursts of exercise expenditure.

### BACKGROUND ART

The stark increase in the “need” by humans to have constant stimulation and information is the object of intense study today in the fields of anthropology and psychology. The craving for stimulation is not new in the human condition, however, recent studies reveal that this craving can manifest in very harmful behaviors, especially among children. A study recently published in the Archives of Pediatrics & Adolescent Medicine states that 1.4% to 17.9% of teens are addicted to the Internet. The study's name is Predictive Values of Psychiatric Symptoms for Internet Addiction in Adolescents, A 2-Year Prospective Study. Leaders of the study are Chih-Hung Ko, MD; Ju-Yu Yen, MD; Cheng-Sheng Chen, MD; Yi-Chun Yeh, MD; Cheng-Fang Yen, MD, PhD. The original study results appear in Arch Pediatr Adolesc Med. 2009;163(10):937-943. Internet addiction early in life can lead to a destructive behavior pattern that may become a lifelong problem leading to an unhealthy sedentary lifestyle.

One therapist concluded regarding this craving for stimulation that:

We humans crave stimulation, and on many different levels. To experience ourselves as fully alive, we all have various “arousal requirements”—whether physical, mental, emotional, or spiritual. And if we feel under-stimulated, we'll generally complain of being bored, antsy, anxious, irritable, lonely, or even depressed. This post explores some of the less fortunate ramifications of our constant need for stimulation. Perhaps more than anything else, our arousal needs—and the negative emotions and states of mind we experience when these needs aren't being met—can interfere with our better judgment. People who suffer accidents and injuries, for example, frequently do so because their personal arousal requirements compel them to take risks ranging from the not-entirely-prudent to the foolhardy or downright reckless. Others may join religious cults or fringe organizations because they're frantically searching for a more fulfilling, spiritual life—but don't really know where to turn. Their need to fit in somewhere, to fill the internal void of un-relatedness, drives them (at times, quite indiscriminately) to put their faith in an ideology, or charismatic leader, that can easily lead them astray.

See Human Nature Abhors a Vacuum Too, 17:00 in Blogs, Psychology Today, Psychologyresearch.or.uk.

Responding to this craving for stimulation and the late twentieth century's new reality of the presence of “spare time,” an entire fitness industry was founded and has evolved into a multibillion dollar industry in the United States alone. The dilemma for the industry is to capture and maintain the attention of its “customers” who are increasingly more demanding for stimulation, for “something new” that will keep them interested in becoming and more importantly remaining fit. Recognizing that many people are really not willing to dedicate themselves to the reality that true exercise is a “workout,” and frankly not easy, many have developed what appears to be a “magic” device that claims that with only five minutes a day of use you will look like the model demonstrating the machine, who in reality probably spends hours in the gym every day. Countless machines have been developed in response to this growing market from treadmills, elliptical machines, stationery bikes, stair stepping machines, ab-rollers, resistance balls, weights, universal machines to pilates reformers and countless others. In addition group exercise classes have been developed with and without the use of devices or weights etc., to appeal to those who most likely would not work out alone and depend upon having a group to provide the stimulation they need to capture and maintain their attention, and in some cases to provide a distraction from the labor of the workout. These classes range from step aerobics, to pedaling on stationery bikes, to aerobic dance, to use of weights.

Aerobics training is a relatively modern phenomenon the genesis of which can be traced to the work of Lt. Colonel (Dr.) Kenneth Cooper¹ in his book Aerobics in 1968, when according to Dr. Cooper only 100,000 people in the United States were jogging. Today Dr. Cooper points out that in the U.S. alone more than 30 million people now jog, while millions more participate in other aerobic forms of exercise. See generally www.cooperaerobics.com. Dr. Cooper's work found global interest and by the 1980s “Aerobics” became the world's most popular fitness activity bar none. Id. ¹Dr. Cooper in 1968 was a flight surgeon and director of the Aerospace Medical Laboratory in San Antonio Tex. Today the Cooper Institute, a 501(c)(3) nonprofit organization founded in 1970, is dedicated in part to wellness through physical fitness involving aerobic exercise, which include group fitness or “aerobics classes.”

Hundreds of millions of people participate in weekly “Aerobics” group fitness classes around the world. By way of a few examples See generally www.lesmills.co.uk. (started in 1980 group exercise to music programs that is currently franchised in more than 100,000 gyms and health clubs in 55 countries with more than 6,000,000 taking classes on a weekly basis); See also www.zumba.com (group exercise to music programs that is franchised in more than 60,000 gyms and health clubs in 85 countries with more than 6,000,000 taking classes on a weekly basis and over 60,000 instructors worldwide; See also www.todays-women-and-health.com/step-aerobics.html (Innovated by Gin Miller around 1989-Step Aerobics continues to be a popular groups fitness “Aerobics” class offered at virtually all gyms and health clubs where group fitness classes are offered).

In the Group Fitness context many participants find that they reach a plateau in their level of aerobic fitness generally measured as the VO2 max which is the highest rate of oxygen consumption attainable during maximal or exhaustive exercise. Wilmore JH and Costill DL. (2005) Physiology of Sport and Exercise: 3^{rd} Edition. Champaign, Ill.: Human Kinetics. After a time period that differs from individual to individual, group fitness participants don't feel as they are getting a “good workout” from the very same class that one time proved very challenging. Studies have shown that vigorous exercise, which is defined as working out at more than 70% of VO2 max, executed by healthy individuals can effectively increase opioid peptides (aka endorphins, a naturally occurring opiate that in conjunction with other neurotransmitters is responsible for exercise induced euphoria and has been shown to be addictive), positively influence hormone production (i.e., increase testosterone and growth hormone), and help prevent neuromuscular diseases. This effect is often the element that causes persons to continue in an exercise program. Gabert, T. E. (1989, April). Attitudes, beliefs, and models in adherence to exercise and sport. Paper presented at the Annual Meeting of the American Alliance for Health, Physical Education, Recreation, and Dance, Boston, Mass. ED 309 160; Goodrick, G. K et al. (1984). Helping adults to stay physically fit: Preventing relapse following aerobic exercise training. Journal of Physical Education, Recreation and Dance, 55(2), 48-49. EJ 296 026.

As a person's aerobic fitness increases (VO2 Max increase) it becomes increasingly more difficult for a person to reach an effort level that achieves the 70% of VO2 max associated with the exercise euphoria often expressed as a “good workout” in a group fitness environment. In most group fitness activities there is an upper limit of aerobic expenditure that a person can experience principally because a group fitness participant's exercise output is tied to the cadence of the music being used and the limits of the various movements made in the group exercise. A person that “does their own thing” in a group fitness environment is most often discouraged from doing so by the instructor teaching the class and at times by fellow classmates and may well be asked not to return to future classes unless they intend to follow the class, it being too distracting to the class otherwise. In an attempt to overcome this exercise plateau and regain the exercise-induced euphoria many will undertake resistance or weight training and intense “burst type” anaerobic activities to increase their VO2 max, however, these have proven to be ineffective. Kraemer W L, Deschenes M R, Fleck S J. Physiological adaptations to resistance exercise implications for athletic conditioning. Sports Medicine 1988 Oct.; 6(4):246-256.

Consequently, over the past 30 years since group fitness has been globally practiced various attempts have been made to increase the energy expenditure in the group fitness environment by designing higher intensity programs, e.g. steps, slides, glider discs, which have been effective in increasing VO2 max in some individuals, however, the same exercise plateau is reached even in these increased intensity programs and for the same reasons, i.e. limitations of body movement and the cadence of the music being used in the class.

As such, the basic concepts of the prior art devices and group classes for exercise stimulation and their uses are disclosed. As stated there are no acceptable devices and methods of use that are able to capture and maintain the attention of a user while simultaneously and consistently providing the means to achieve 70% of VO2 max, the perceived level of a “good workout” resulting in a concomitant endorphin surge.

While each of these prior art examples disclose exercise devices and group fitness classes which fulfill their respective particular objectives and requirements, and are most likely quite functional for their intended purposes, it will be noticed that none of the prior art cited disclose an apparatus and/or method that allow a user the ability to safely increase both caloric and aerobic expenditure either working out alone or in a group setting with a device that is interactive, making use of a distinct audible indicator, and able to monitor and control the fitness environment in real time according to the needs of the user. As such, there apparently still exists the need for a new and improved exercise device and method of use to maximize the benefits to the user and minimize the risks of injury from its use. In this respect, the present invention disclosed herein substantially fulfills this need.

A significant problem with the prior art devices and group fitness classes is that a user or participant has no meaningful means of monitoring their effort level in an objective manner or to receive self actuated rhythmic stimulation. In this respect, the present invention disclosed herein substantially fulfills this need.

### DISCLOSURE OF THE INVENTION

In view of the foregoing limitations inherent in the known types of exercise devices and group fitness programs now present in the prior art, it is an object of the present invention to combine a percussive type instrument, an electronic monitoring and rhythmic control device with free weights in a novel exercise device adapted to be worn as a garment on various parts of the body without engaging the muscles of the hand for the purpose of increasing the muscle load to improve a workout, incorporating live percussion instrumentals into a private or group workout, to avoid sports injury associated with holding weights directly by hand, and to provide a method of conducting an exercise program which teaches the user of the present invention, through percussion, how to achieve the proper form of a given exercise movement. Additionally the present invention will monitor a range of user, instructor or physician selected “life signs” and according to a user, instructor or physician selected or created exercise program the device can alert the user to change cadence to achieve a particular level of output or it can by means of an electronic interface pitch up or down the tempo of the music being used as the pace of his exercise until his body reaches the desired level of exertion as calculated by real time monitoring as opposed to unreliable perceived levels of exertion which often are psychologically driven and are not routed in actual output of the body. Unreliable perceived levels of exertion are the standard used typically in today's group fitness exercise setting, and the primary accepted method of attaining participant exertion levels during a group exercise class, as taught by nationally certifying aerobic and personal training instructor training programs such as ACE, AAFA, WITS (World Instructor Training Schools whose courses are taught in a university setting as opposed to a weekend certification program site.) Alternatively, many instructors are taught by these national fitness organizations to interrupt their fitness class to ask the participants to take their own pulse manually by watching a second hand on a clock in the workout studio, if one is even available, and take their pulse for 6 seconds and multiply by 10 for heart rate per minute. This form of measurement is fraught with inefficiencies and inaccuracies as individuals may have difficulty locating their pulse, once located recording it accurately, using exactly 6 seconds, and so on. One clinical study of particular interest to the present invention demonstrating how subjective and inaccurate can be the perceived level of exertion relative to objective measurement of vital signs, Physiological Responses to walking with hand weights, wrist weights and ankle weights, Med. Sci. Sports Exerc. 1988 Jun.; 20(3):265-71, compared the blood pressure (BP) response to exercise with 1.36 kg (3.0 lb hand-held weights (HW), wrist weights(WW), and ankle weights (AW). The study demonstrated that while oxygen uptake and heart rate responses were statistically significantly greater for HW, WW, and AW than exercise with no weights (NW), “ratings of perceived exertion (Borg scale for NW (11.7+/−1.8), HW (12.1+/−2.0). WW (12.3)+/−1.8) were not significantly different (P greater than 0.05)” This study demonstrates two pertinent points: 1) that although exertion was significantly greater wearing and using weights, the perceived level of exertion was not; and 2) that contrary a widely held perception in the fitness industry that worn weights do not significantly increase the cardiovascular challenge, worn weights do in fact increase the work demand during exercise and as such can help overcome the exercise plateau syndrome. In the fitness industry today, it is widely accepted, although not clinically supported that hand held, wrist worn, and standard weights used in group fitness activities generally will not improve a person's VO2 max, simply because the speed of movement is often slowed as weight is added or the amount of weight may not be enough to increase cardiovascular system demand significantly. Contrary to “myth”, Bhambhani Y, Burnham R, Singh M, Gomes P, Faculty of Rehabilitation Medicine at the University of Alberta, Edmonton, Canada, as published in Am J Sports Med. 1987 September-October: 15(5):508-10 concluded “Energy expenditure and heart rate increased as a linear function to the additional weight placed at both anatomic locations [wrist and ankle] . . . . Since ankle and wrist weights increase training intensity and energy expenditure during treadmill running, they may result in greater increases in cardiovascular fitness and greater weight loss than would be realized by training without their use.” Miller J F, Stamfor B A, in a study entitled “Intensity and energy cost of weighted walking vs. running for men and women” published in J Appl Physiol. 1987 April; 62 (4); 1497-501 found that “Intensity of effort and energy cost per minute and per mile were increased when weight was added during walking and were increased more with hand weights compared with ankle weights regardless of speed . . . . The intensity of walking at 4 mph with ankle and hand weights was comparable to running at 5 mph.” As such, the general purpose of the present invention, which will be described subsequently in greater detail, is to provide a field designed apparatus and method of use that incorporates the present invention. There are many additional novel features directed to solving problems not addressed in the prior art.

To attain this the present invention generally comprises a “vital signs” electronic monitoring device; a music source in electronic communication with the “vital signs” electronic monitoring device such that the type of musical sound, volume and/or speed of the music are automatically adjusted in response to the user's vital signs and the user's, instructor's or physician's pre-selected exercise output level; a percussive type instrument; weights; and a garment capable of securely holding the monitoring and electronic device, instrument and weight. In certain embodiments the vitals monitor may be located separately from the rest of the components contained in the garment, and the vitals may be transmitted to the electronic device connecting and interpreting the data.

Several objects and advantages of the present invention are:

There have been numerous studies that indicate that the use of hand held weights greater than one pound in repetitive motions during exercise has lead to various injuries such as carpal tunnel syndrome, tendonitis, DeQuervain's disease and ECU tendonitis. See Body building-effect on neural conduction velocity of the median nerve in carpal tunnel, [Article in German] Mauer U M, Lotspeich E, Klein H J, Rath S A, Z Orthop Ihre Grenzgeb. 1991 July-August:129(4):319-21 and Stress-induced carpal tunnel syndrome in athletes-exemplified by 3 kinds of sports [Article in German] Mauer UM, Rath S A, Schweiz Z Sortmed. 1992 September: 40(3)131-5. The present invention is specifically designed and field tested to permit a user to add weight during aerobic exercise that does not require the weight to be held in the hand and is positioned upon a user in such a way as to virtually eliminate the risk of these injuries. During the 1980s a trend developed within gyms and health clubs where group fitness participants would use hand and ankle weights greater than one pound during group fitness classes to overcome this exercise plateau and increase VO2 max. However, due to the lack of a device such as the present invention the incidence of injury from the unstudied and undeveloped use of significant hand held weight loads was so pronounced that AFAA, ACE, IRSHA, the Cooper Clinic and other group fitness instructor certification organizations all but universally banned the use of significant weights during group fitness activities and most certified instructors will require group aerobic fitness participants stop using them in class if one attempts to do so.

Although there is clinical evidence to support the value of an additional muscle load by using weights during aerobic exercise with respect to cardiovascular challenge, there has not yet been consensus on how to harness the benefit of that challenge versus the risk of injury. Although widely respected, renowned pioneers in sports- and dance-medicine, as well the younger generation of orthopedic surgeons in sports medicine have been willing to medically endorse the use of low weight worn wrist and ankle weights during aerobic exercise, there, unfortunately, exists an industry “taboo” against all hand-held and worn weights, no matter their weight, or low risk of injury, no matter that evidence-based medicine as clinically supports their use. (James G. Garrick, MD, Orthopedic Surgeon and Medical Director of the James G. Garrick Centers for Sports Medicine, Professor, University of California, San Francisco Medical School, pioneer and expert in the etiology of dance injury, published studies 1986 and personal conversation with the inventor, February, 2009; Susan Lewis, MD, Orthopedic Surgeon, Emergency Medicine, Faculty University of California, San Francisco Medical School, The James G. Garrick Centers for Sports Medicine, meeting with inventor and Bruce B. Feinberg MD, Harvard Medical School, March 2010.)

It has also been observed that accidents have occurred when hand weights during extreme movements of group fitness activities have come free from the grip of a user posing an unacceptable risk to others in the group fitness class. Kraemer W L, Deschenes M R, Fleck S J. Physiological adaptations to resistance exercise implications for athletic conditioning. Sports Medicine 1988 Oct.; 6(4):246-256. The present invention overcomes this problem since the weights are securely attached to the user by various methods that do not require a user to maintain a grasp on the weight, which is of course most important during latter portions of a user's exercise as the user fatigues and more likely to lose their grip.

The present invention specifically teaches and claims the use of weights during exercise to music, such as in a group fitness environment, which heretofore was deemed impossible to be done safely. At first blush this would appear to contradict the current convention that specifically recommends against the use of weights at all. However. current convention is not based in evidence-based medicine, which supports the use of light weights (one pound or less). In fact, regarding the use of one-pound bracelet style wrist weights, based on a ten week study at the Center for Sports Medicine-Dance Medicine, Dr. Garrick told the third annual International Dance-Exercise Assn Convention, “We have . . . seen no evidence that the use of wrist weights results in any increase in injuries . . . . The injury rate is nearly identical to that seen in the original study in which the weights were not used . . . ” Furthermore, in conclusion Garrick said for those individuals who are “maxed out on the class they're in”—who are too fit for their current class level and aren't showing any more improvement—the use of wrist weights can enhance the workout level.” See Los Angeles Times News, Jun. 3, 1986 Wrist Weights Not Linked to Injuries . . . by Dennis Mclellan, Times Staff Writer. And as noted, the inventor has confirmed personally with Dr. Garrick in a phone conversation in February 2009 that he still holds by his 1986 study and would claim the same for use of one pound ankle weights. Thus in distinction to convention but not contrary to science, the present invention, provides a means of allowing the addition of a minimal amount of weight sufficient to increase a person's energy output without slowing the movement of limbs and torso during group exercise and minimize the risk of injury at the same time, which is a long felt need in the group fitness industry. The weighted musical device is worn and not held and as such can be located on a user's body nearer to the body core and gravitational center which reduces, if not eliminates, any risk of injury to joints.

The present invention effectively combines four distinct means by which one can overcome an exercise plateau and maintain interest in physical exercise. The first being added weight that is of a size and location on a user that would not slow body movements, but would permit a user to “keep up with the music.” Secondly, by adding weight which is worn, not held, in a manner which distributes the weight evenly throughout the entire device, as in a sleeve, on the calf, thigh, around the hips, and arms, the risk of injury which deterred the fitness industry from engaging in strapping on weights specifically at the joints is reduced or rendered negligible. See www.bodytogs.com. Thirdly, the present invention discloses a device that allows a user the added benefit of providing a movement activated rhythmic accompaniment to the music being used during exercise. The interactive nature of the device as claimed herein further involve the user in the music and thereby inspiring a user to keep up to tempo with the music. Lastly, the device incorporates interactive “vital signs” monitoring. In one embodiment the user can enter their age and fitness parameters into the electronic monitoring device and select an exercise type, such as a standard aerobics program, i.e. warm up—workout—cool down or perhaps an interval training program. The electronic monitoring device can audibly alert the user as to what phase of the workout they are in and the user will then adjust their output accordingly. In yet another embodiment the electronic monitoring device will automatically adjust the speed, or pitch, or type of music that the user may be using as a tempo for the pace of their exercise and the rhythmic accompaniment of the device in order that the user's exercise output as it seeks to match the changed cadence of the music will reach the user, instructor or physician selected “life sign” parameters, which could be one or more of the following singly or in any combination with the others such as the user's pulse, blood pressure, saturated blood oxygen level, body temperature, respiratory rate and percentage of VO2 max.

The group fitness aerobics exercise plateau problem described above and highlighted in the cited references has manifested in a long felt need for a device and method of use such as the present invention. It is a problem that has been worked on for at least the last thirty years by the Group Fitness industry as a whole and most recently by the world's leading group fitness franchise program Zumba®. Les Mills™, the world's second largest group fitness franchise has focused solely on using up tempo music and highly motivated and trained instructors to bring its aerobics class participants to 70% of their VO2 max and has resigned itself to use weights only in more controlled anaerobic exercise directed at body toning which does not increase VO2 max and solve the aerobic exercise plateau problem. Suffice it to say that Les Mills™ aerobics classes have not overcome the group fitness aerobics exercise plateau problem. Kraemer W L, Deschenes M R, Fleck S J. Physiological adaptations to resistance exercise implications for athletic conditioning. Sports Medicine 1988 Oct.; 6(4):246-256.

Further evidence of the novelty and industry need of the present invention, is demonstrated definitely, in the offer of employment to the inventor by Zumba® Fitness as Assistant Vice President of Marketing, and her current position as Public Relations Representative for Zumba® Fitness. The inventor of the present invention was not known to Zumba® except for her presentation of her invention to the creative founder Beto Perez immediately after filing the current patent application, and her subsequent invitation by the CEO, Alberto Perlman based on the recommendation of Beto Perez. Furthermore, the inventor of the present invention has entered into a non-disclosure agreement with Les Mills™ (with first rights of refusal granted to Zumba® Fitness, and the current invention is presently under review by Vaughan Schlass, Director of Marketing for Les Mills™, the in-house medical team, and choreographers for potential co-launch in their new basic dance fitness program. Moreover, the inventor of the present invention has signed two contracts with Latin Percussion™, the largest manufacturer of hand-held shaker instruments in the world, with the greatest number of patents in the music industry for innovative shaker instruments. Additionally, the inventor of the present invention has been hired as a public relations consultant based on her high level connection: in the fitness industry; in Washington, D.C., including the President's Council for Physical Fitness and Sports; and her consistent communication with the new Executive Director Shellie Pfohl ever since her presentation February 2010 of percussive fitness in combination with Zumba® Fitness as an effective new form of physical activity in the challenge to overcome childhood obesity. The inventor of the present invention was invited by the Office of the First Lady to the White House in February 2010 to attend the First Lady's exclusive launch of the Let's Move Initiative. As well, the inventor of the present invention has signed a contract with Latin Percussion, Inc. to produce the invention, bringing it to market within two years. These facts clearly support the industry long felt need being met by the present invention, as evidenced by its inventor's sudden rise to the highest levels of the fitness and music industries, and recognition by the First Lady.

These together with other objects of the invention, along with the various features of novelty which characterize the invention, will be pointed out with particularity in the claims which are annexed to and form a part of this patent application. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The above specification, as well as further objects, features, advantages and embodiments of the present invention, will be more fully appreciated by reference to the following detailed descriptions, when taken in conjunction with the accompanying drawings, wherein:

FIG. 1 illustrates a front view of a first embodiment of a weighted exercise percussive device according to the present invention, worn in an encircling position around forearm and consists of a form fitting, stretchable sleeve (labeled A) with at least one sealable pocket (only one pocket is necessary if the shaker and weight are combined into one object. See FIG. 23). The visible front pocket (labeled B) will hold a weight and/or a shaker instrument, which is retained in place by a seal such as Velcro, zipper or snap (labeled C).

FIG. 1A illustrates one of many possible percussive type instruments, in this case a cross-sectional view of a semi-flat disc containing noise making material therein (labeled D), to be inserted into one of the said pockets (labeled B).

FIG. 2 illustrates a posterior view of the weighted exercise percussive device depicted in FIG. 1 encircling the forearm as a sleeve, consisting of a form fitting, stretchable material such as Spandex (labeled I) with elastic rim (labeled H) with two sealable pockets. The visible underside compartment (labeled F) will hold a weight and/or a shaker instrument or the combined weighted-percussive exercise device, which is retained in place by a seal such as Velcro, zipper or snap (labeled E).

FIG. 2A illustrates one of many possible weights (labeled G) to be inserted into one of the said pockets, in this case a flexible pouch filled with material such as lead, steel, tungsten, sand or gel.

FIG. 3 illustrates a frontal view of an embodiment of a weighted exercise percussive device according to the present invention, variable to the size of the wearer consisting of an open ended stretchable material intended to encircle a body part, such as (but not limited to) the wrist, forearm, ankle, or leg, which is open at both ends like an adjustable belt, making use of a closure mechanism such as Velcro (labeled C). This embodiment has at least one pocket (labeled B), and each pocket is opened and sealed by any common means such as Velcro, snaps, or hooks (labeled A) used to retain a shaker and a weight, or the embodiment of the present invention which is both a shaker and a weight simultaneously; that is, the combined weighted-percussive exercise device.

FIG. 4 illustrates an embodiment of a weighted percussive exercise device according to the present invention, variable to the size of the wearer consisting of an open ended stretchable material intended to encircle a body part, such as (but not limited to) the wrist, forearm, ankle, or leg, which is open at both ends like an adjustable belt, making use of a closure mechanism such a Velcro strap (labeled A) threaded through a buckle (labeled B). This embodiment has at least one pocket, and each pocket is opened and sealed by any common means such as Velcro, snaps, or hooks, used to retain a shaker or a weight, or the combined weighted-percussive exercise device of the present invention.

FIG. 4A is an enlarged view of the buckle (labeled B) and the Velcro straps (labeled A) which are threaded through that buckle from both sides and then folded back onto the strap, adhering by Velcro.

FIG. 5 illustrates an embodiment of the weighted percussive exercise device according to the present invention, worn in an encircling position around the upper arm, such that it consists of a form fitting, stretchable sleeve (labeled A) with at least one sealable pocket (labeled B) each for a shaker, weight, or the combined weighted-percussive exercise device of the present invention, which is retained in place by a seal such as but not limited to Velcro, zipper or snap (labeled C).

FIG. 6 illustrates a frontal view of an embodiment of the weighted exercise percussive device according to the present invention, worn in an encircling position around the wrist, such that it consists of a form fitting, stretchable wristband (labeled B) with at least one sealable pocket (labeled A) each for a shaker, weight, or the combined weighted-percussive exercise device of the present invention, which is retained in place by a seal such as Velcro, zipper or snap.

FIG. 7 illustrates a posterior view of an embodiment of the weighted percussive exercise device according to the present invention, worn in an encircling position around the wrist such as it is in FIG. 6 with the addition of a loop around the thumb (labeled D) to stabilize the said device against movement, such a loop being common in the fitness and rehabilitation industries for products wrapped around the wrist, such that it consists of a form fitting, stretchable wristband (labeled E) with at least one sealable pocket (labeled C), each pocket used for a shaker, weight, or the combined weighted-percussive exercise device of the present invention, which is retained in place by a seal such as Velcro, zipper or snap.

FIG. 8 illustrates a frontal view of an embodiment of the weighted exercise percussive device according to the present invention, worn in an encircling position around the abdomen, secured with an adjustable closure such as a Velcro strap (labeled A) and buckle (labeled B), containing at least one sealable compartment (labeled C) each used for a shaker, weight or the combined weighted-percussive exercise device of the present invention, which is retained in place by a seal such as Velcro, zipper or snap.

FIG. 9 illustrates a frontal view of two embodiments of the weighted exercise percussive device, according to the present invention worn in an encircling position around the ankle. The first of these embodiments, (labeled A) illustrates an ankle only encirclement, which can be secured with an adjustable closure such as a Velcro strap and buckle, or as an elasticized band that slides over the foot (labeled B). In the second embodiment the weighted percussive exercise device is worn like a sock/ankle brace, such that the stretchable sleeve encircles both the ankle and upper foot (labeled C), with a cutout for the heel (labeled D). Both embodiments contain at least one sealable compartment (labeled D) each compartment used for a shaker, weight or the combined weighted-percussive exercise device of the present invention, which is retained in place by a seal such as Velcro, zipper or snap.

FIG. 10 illustrates a frontal view of two embodiments of the weighted exercise percussive device, according to the present invention worn in an encircling position around the leg. Each embodiment consists either of a form fitting, stretchable sleeve pulled over the foot and worn as a band, or as an adjustable belt like the device depicted in FIG. 3, encircling the thigh (labeled A) or the calf (labeled D); each embodiment containing at least one sealable compartment (labeled B, C, E and F), each used for a shaker, weight or the combined weighted-percussive exercise device of the present invention, which is retained in place by a seal such as Velcro, zipper or snap.

FIG. 11 is a dorsal view of the weighted percussive exercise device, according to the present invention consisting of a form fitting, stretchable sleeve worn in an encircling position around the hand, such that the device is secured by any common closure material such as a Velcro strap (labeled A) threaded through a buckle (labeled B); so that the strap then folds back over itself (labeled C), and containing at least one sealable compartment, each used for a shaker, weight or the combined weighted-percussive exercise device of the present invention, which is retained in place by a seal such as Velcro, zipper or snap.

FIG. 12 is a ventral view of the weighted percussive exercise device, according to the present invention consisting of a form fitting, stretchable sleeve worn in an encircling position around the hand, containing sealable compartments, in this case three (labeled A), each compartment used for a shaker, weight, or the combined weighted-percussive exercise device of the present invention, which is retained in place by a seal such as Velcro, zipper or snap, and secured around the hand by any such means of closure such as an adjustable Velcro strap (labeled B).

FIG. 13 illustrates a frontal view of an embodiment of the weighted exercise percussive device, according to the present invention, consisting of a form fitting, stretchable sleeve or adjustable belt, secured and retained by any such means of closure such as an adjustable Velcro strap or elastic band, worn in an encircling position around the torso containing at least one sealable compartment, in this case five (labeled A), each compartment used for a shaker, weight or the combined weighted-percussive exercise device of the present invention, which is retained in place by a seal such as Velcro, zipper or snap.

FIG. 14 illustrates a lateral view of an embodiment of the weighted percussive exercise device, according to the present invention, worn in an encircling position around the foot, consisting of a form fitting, stretchable sleeve which slides on like a sock (labeled A), secured by any common means of closure such as an adjustable Velcro strap or elastic band (labeled B), containing at least one sealable compartment (labeled C), each used for a shaker, weight or the combined weighted-percussive exercise device of the present invention, which is retained in place by a seal such as Velcro, zipper, snap or elastic band.

FIG. 15 illustrates a dorsal view of an embodiment of the weighted percussive exercise device, according to the present invention, worn in an encircling position around the foot, consisting of a form fitting, stretchable sleeve which slides on like a sock (labeled A), secured by any common means of closure such as an adjustable Velcro strap or elastic band, containing at least one sealable compartment (labeled B), each used for a shaker, weight or the combined weighted-percussive exercise device of the present invention, which is retained in place by a seal such as Velcro, zipper, snap or elastic band.

FIG. 16 illustrates a frontal view of an embodiment of the weighted percussive exercise device, according to the present invention, worn as a form fitting shirt of stretchable material like Spandex (labeled B), containing sealable compartments sewn directly into the fabric, in this view, one such compartment in the center front (labeled A), each compartment used for a shaker, weight or the combined weighted-percussive exercise device of the present invention, which is retained in place by a seal such as Velcro, zipper or snap (labeled C).

FIG. 17 illustrates a posterior inside/out view of the same embodiment of the weighted percussive exercise device, depicted in FIG. 16, according to the present invention, worn as a form fitting shirt of stretchable material, containing sealable compartments, in this view, two such compartments (labeled B) under the shoulder blades, each for the purpose of containing a shaker, weight or the combined weighted-percussive exercise device of the present invention, which is retained in place by a seal such as Velcro, zipper or snap, (labeled A).

FIG. 18 illustrates a frontal view of a embodiment of the weighted percussive exercise device, according to the present invention, worn as a form fitting sport bra of stretchable fabric such as spandex, containing at least one sealable compartment (labeled C), each for the purpose of containing a shaker, weight or the combined weighted-percussive exercise device of the present invention, which is retained in place by a seal such as Velcro, zipper or snap, or secured by any common means of closure (labeled B). In this depiction, one compartment is sewn into the center front (labeled A).

FIG. 19 illustrates a posterior view of an embodiment of the weighted percussive exercise device, according to the present invention, worn as a form fitting sport bra of stretchable fabric such as spandex, containing at least one sealable compartment; in this view, two such compartments under the shoulder blades (labeled A), each for the purpose of containing a shaker, weight or the combined weighted-percussive exercise device of the present invention, which is retained in place by a seal such as but not limited to Velcro, zipper or snap (labeled B).

FIG. 20 illustrates a lateral view of an embodiment of the weighted percussive exercise device, according to the present invention, such that weights, shakers or combined weighted-shakers are inserted into fitted, sealable pockets sewn into trousers or shorts made either of stretchable, form fitting fabric such as spandex, or loose garment (labeled B). Depicted in FIG. 20 are three such pockets, one on the upper leg, one on the calf (labeled A), and one in the back on the buttocks (labeled C), each for the purpose of containing a shaker, weight or the combined weighted-percussive exercise device of the present invention, which is retained in place by a seal such as Velcro, zipper, snap, rivet, or elastic band (labeled D).

FIG. 21 illustrates a lateral view of an embodiment of the weighted percussive exercise device, according to the present invention, worn as a headband (labeled A and A1) such that weights, shakers or combined weighted-percussive device are inserted into at least one fitted, sealable pockets (labeled B), secured by any typical means of closure such as Velcro, snap, rivet, or zipper.

FIG. 22 illustrates a lateral view of an embodiment of the weighted percussive exercise device, according to the present invention, consisting of a form fitting, stretchable sleeve with an opening at each end (labeled A), designed to be worn around a shoe which slides on like a sock, or secured like a belt adjusted with Velcro. In both embodiments, the device contains at least one compartment (labeled B), each for the purpose of containing a shaker, weight or the combined weighted-percussive exercise device of the present invention, which is retained in place by a seal such as but not limited to Velcro, zipper or snap.

FIG. 22A depicts a shoe hosting the embodiment of the weighted exercise percussive device (labeled B), according to the present invention, illustrated in FIG. 22, secured in this case with elastic bands (labeled A).

FIGS. 23, 23A, 23B, 23C, 23D, and 23E are significant over FIGS. 1-22A because each of FIGS. 23, 23A-E illustrates a cross-sectional view of an embodiment of the weighted percussive exercise device approximating actual size, according to the present invention, whereby, rather than a separate weight and separate shaker instrument housed in separate designated compartments, herein the shaker instrument and weight are combined into one object; that is, a weighted-percussive exercise device, such that the shaker assembly consists but is not limited to a hard container of various possible shapes: specifically FIG. 23 is rectangular; FIG. 23A is oval; FIG. 23B is circular; FIG. 23C is cylindrical; FIG. 23D is triangular; and, FIG. 23 E is a wave, being chosen to emphasize that the essential character of this particular embodiment of the invention is not the shape of the container, which is actually quite variable, but rather, it is the combined percussive, precisely weighted contents thereof and therein; such that the weight and shaker instrument are not two separate objects.

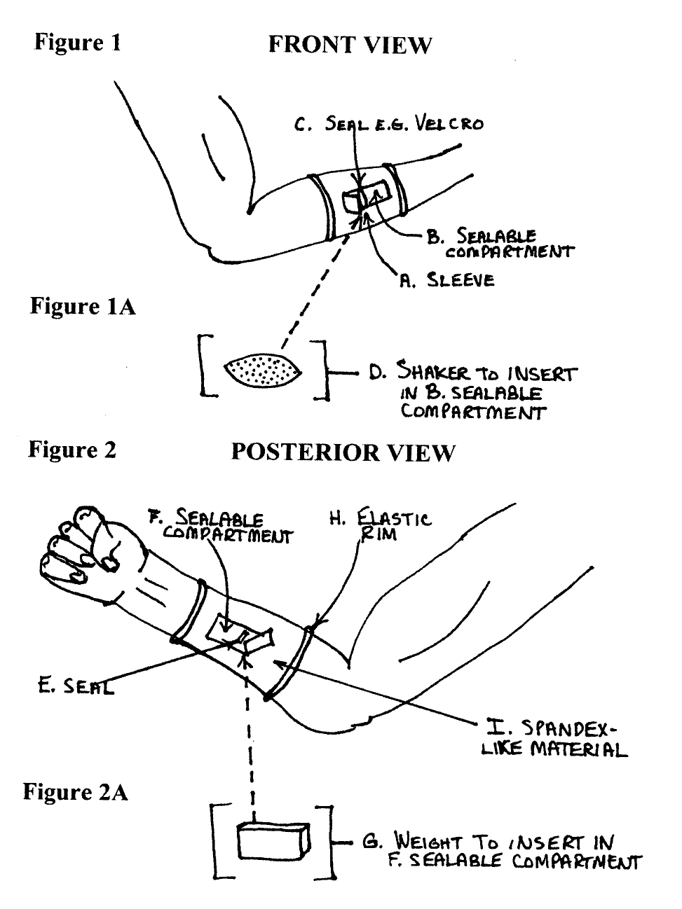

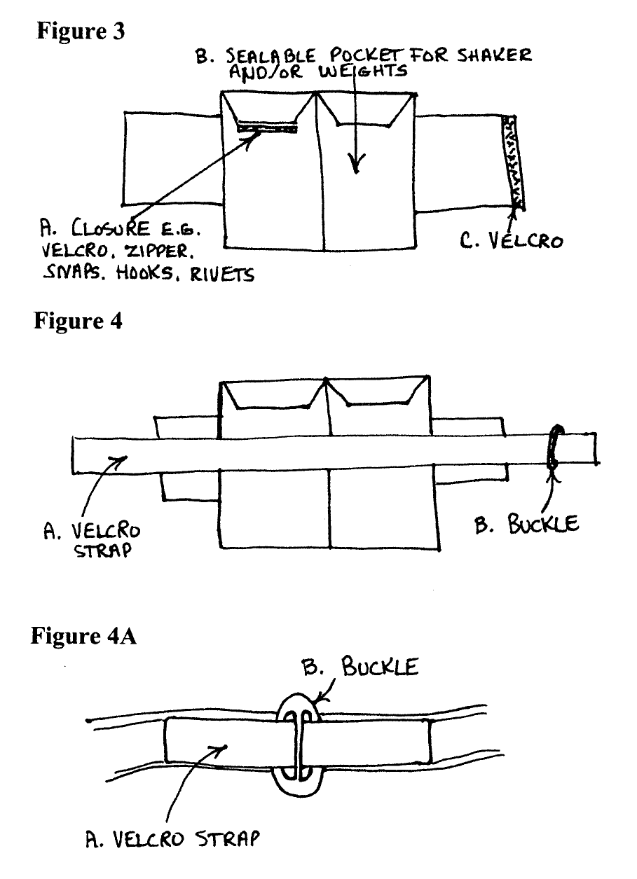

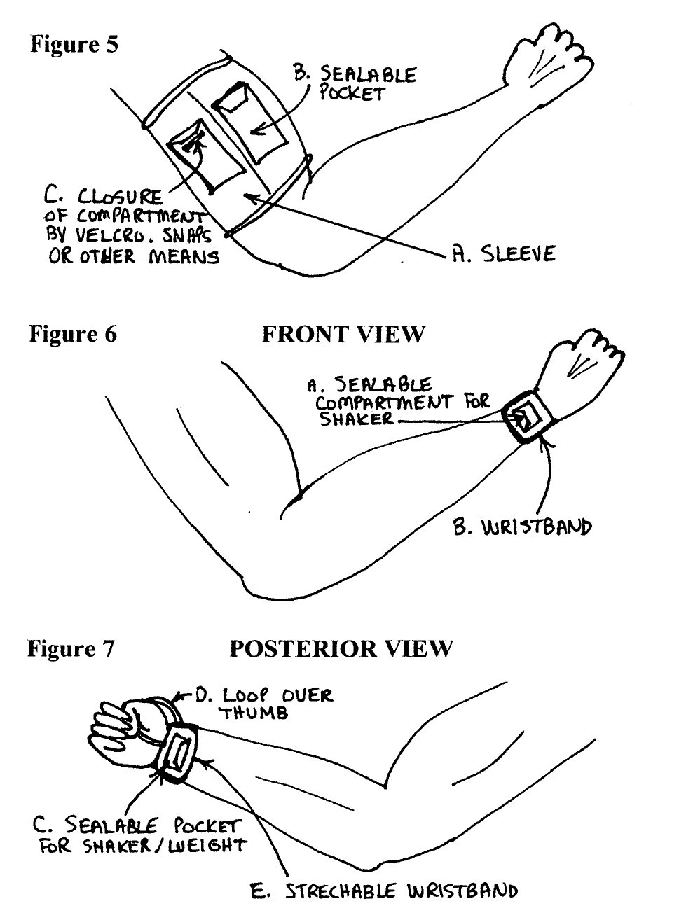

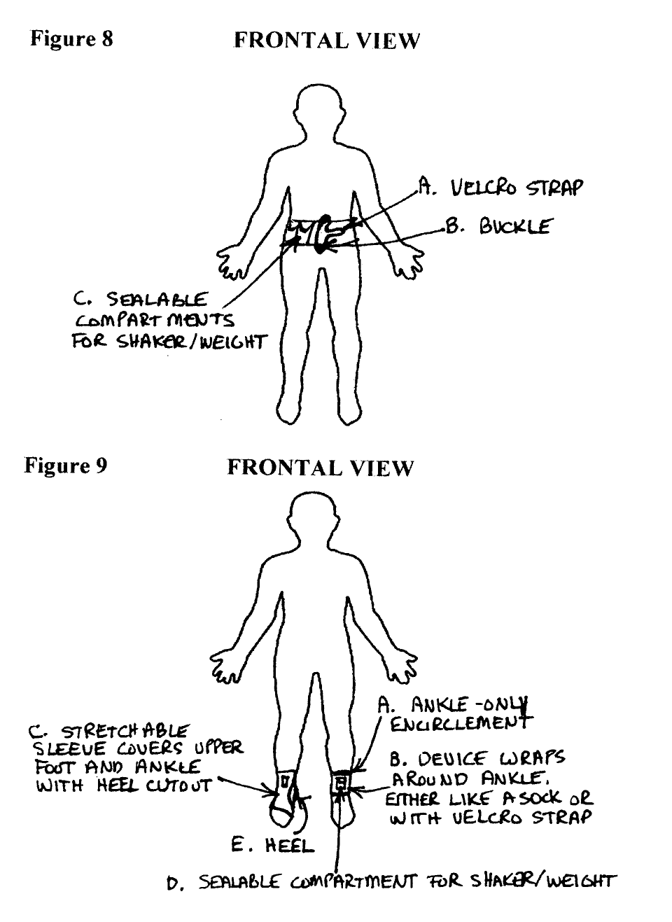

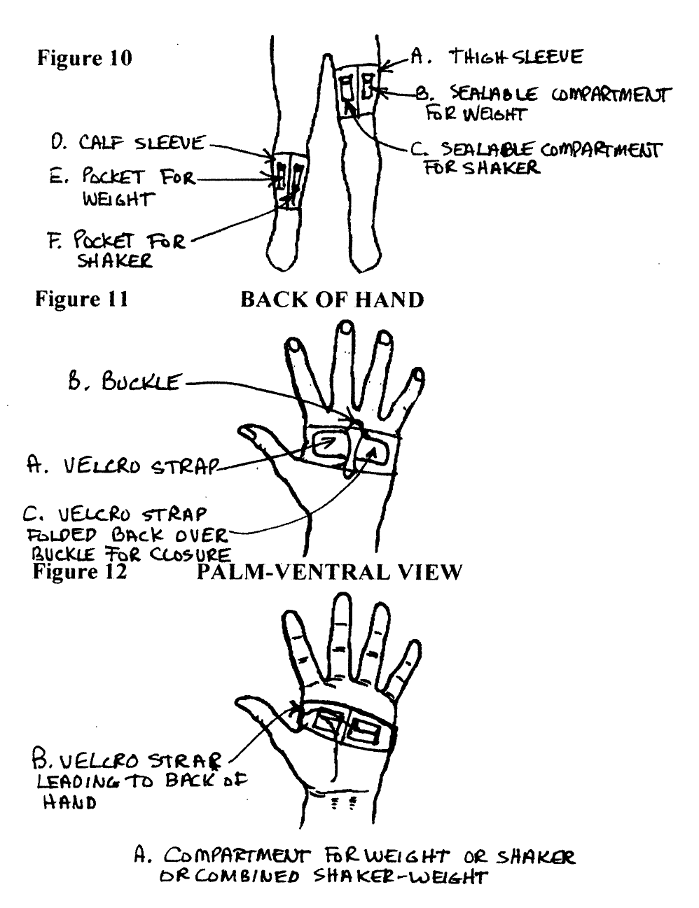

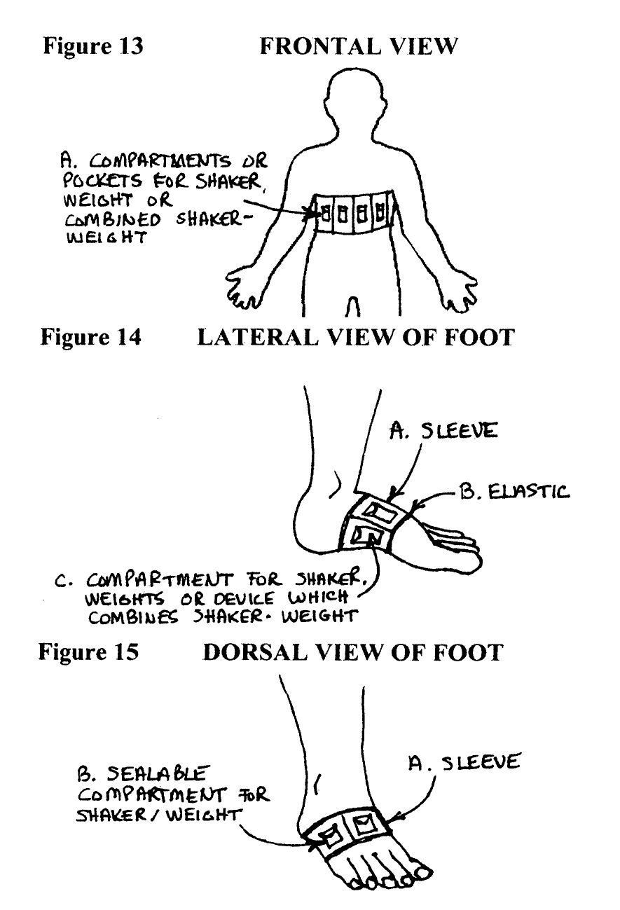

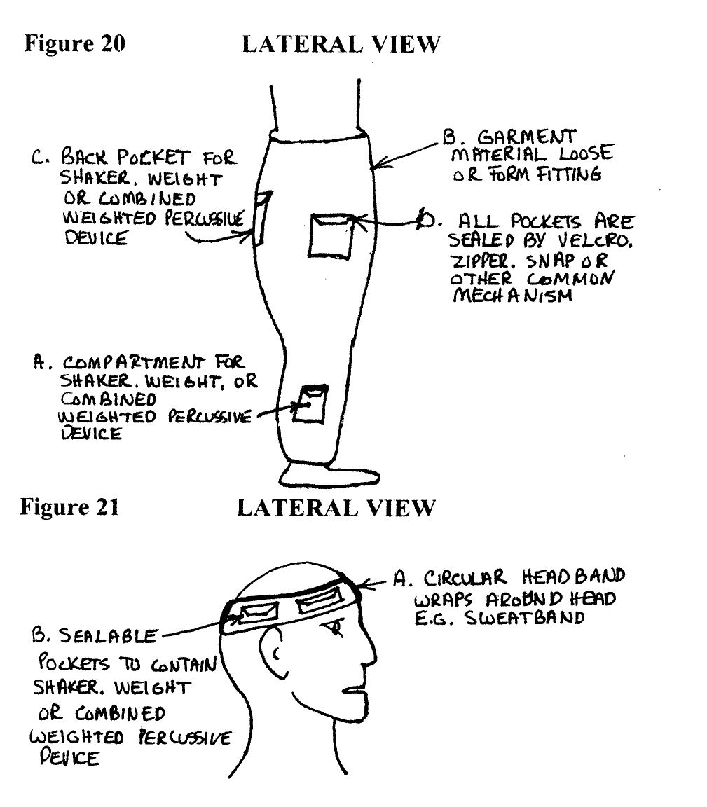

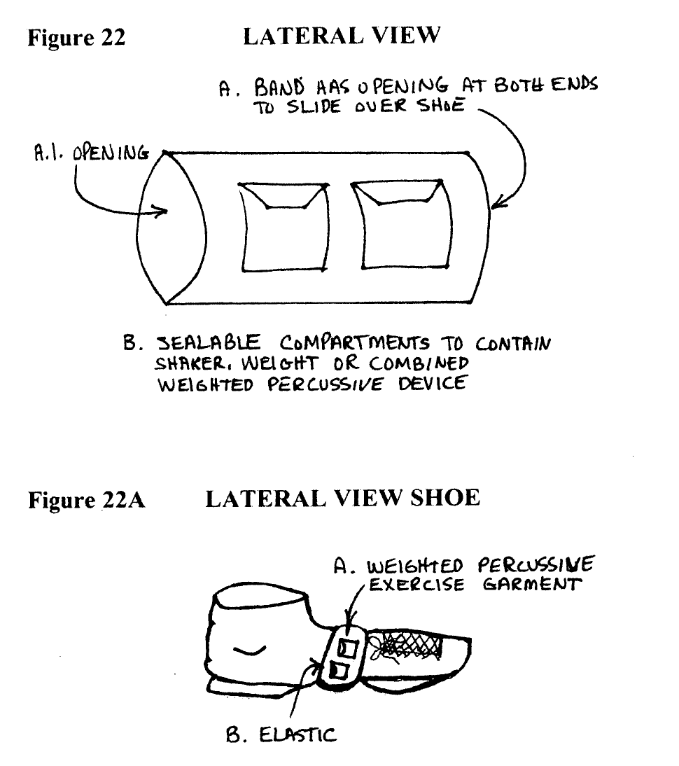

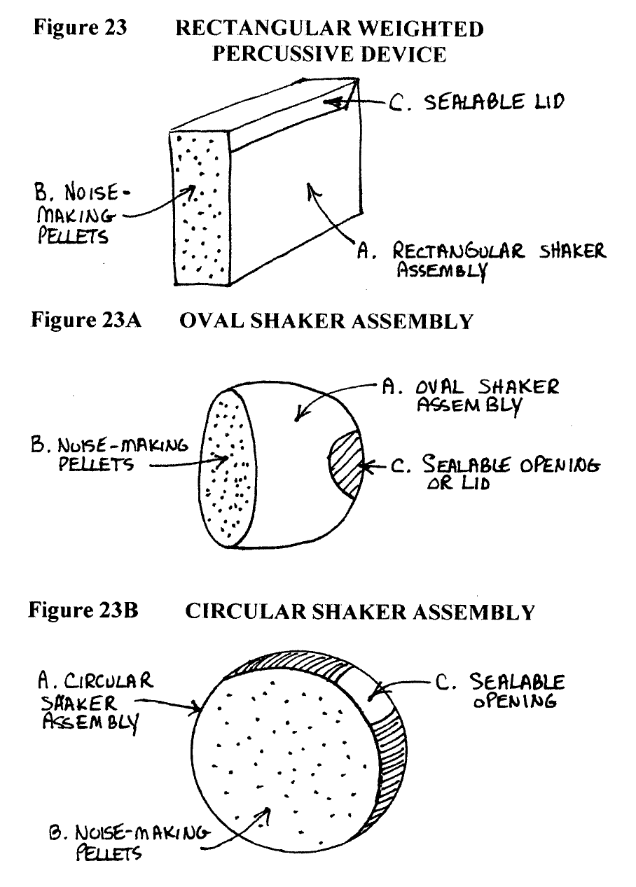

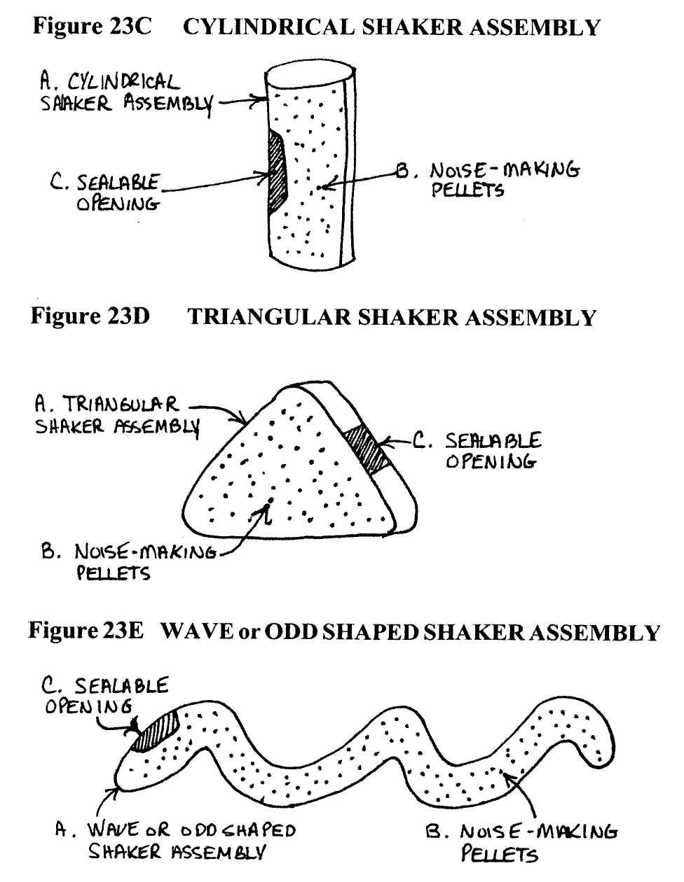

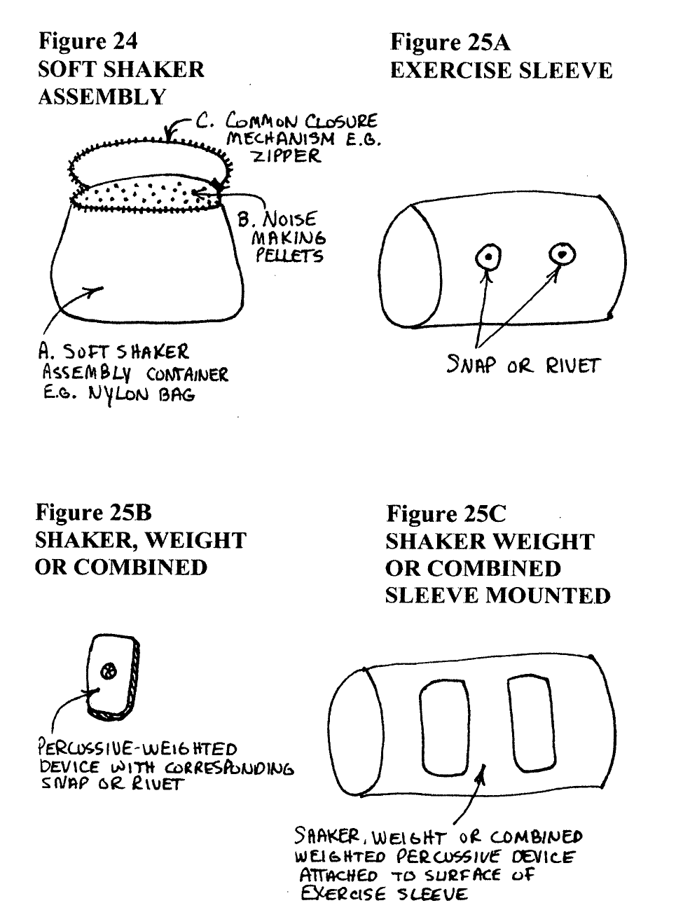

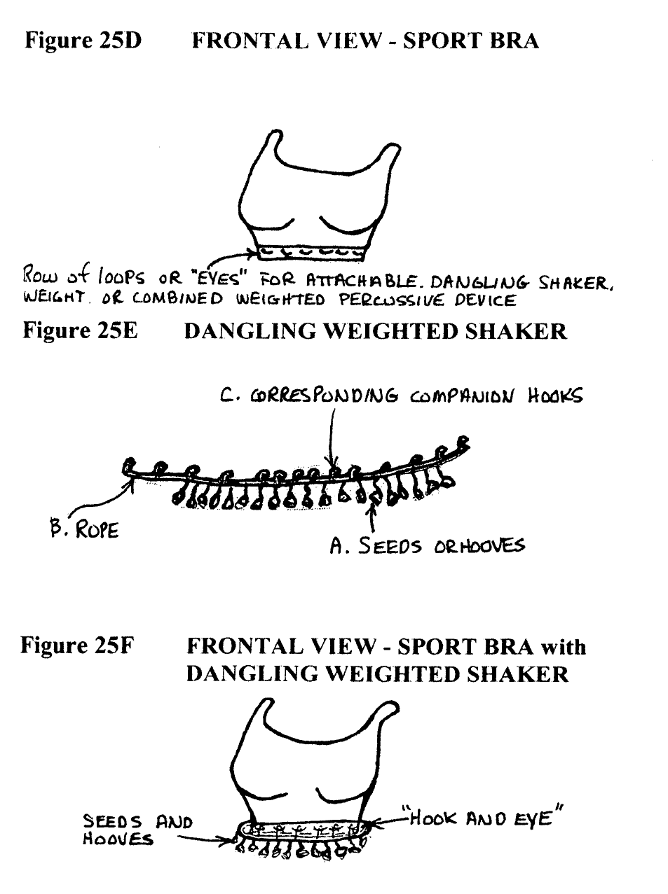

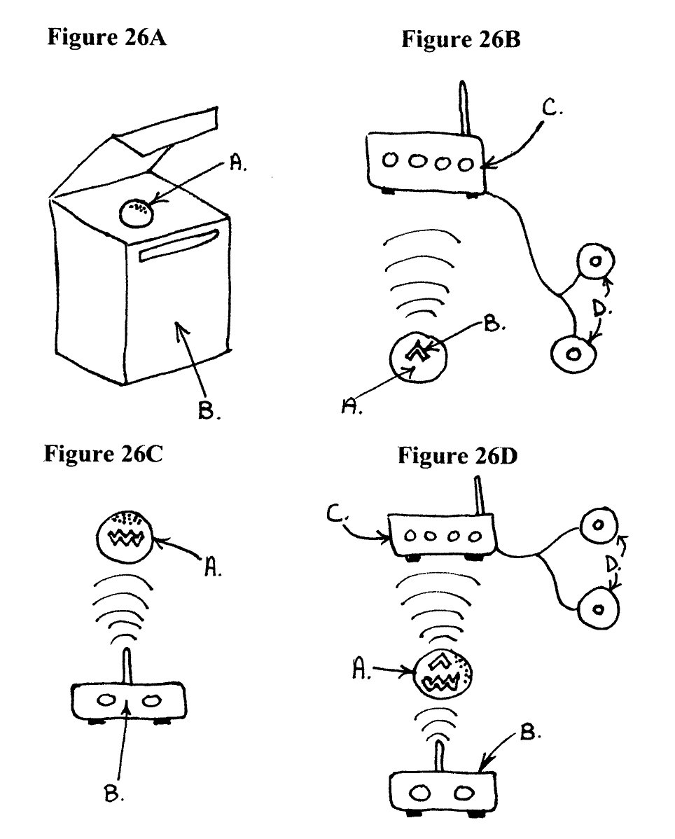

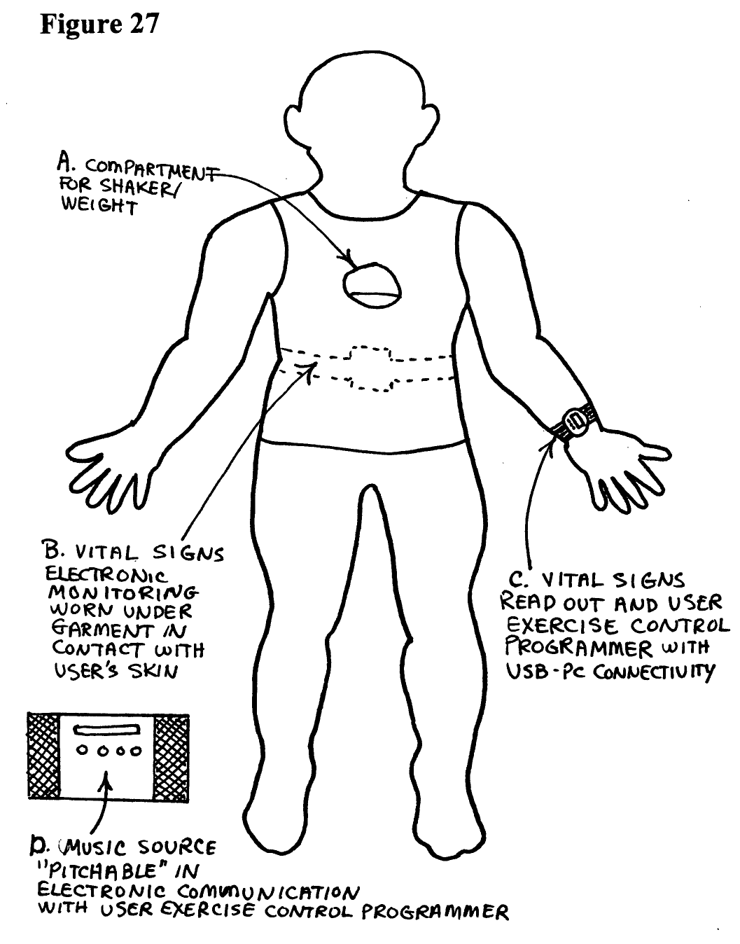

Each such embodiment FIGS. 23, 23A-E is enclosed with a sealable lid (labeled C), and has located therein a plurality of interchangeable hard pellets, balls or bells formed from a weighty material such as tungsten, plastic, steel, lead, or other noisemaking material (labeled B), which can be removed in exchange for other pellets in order to change the quality of the percussive noise; the quantity of which are also interchangeable so that the quantity amounts to specifically desired weight intervals which can be changed in order to add or decrease weight; so that the pellets, balls or bells of desired 1) noise quality and 2) weight are free to move about within the container for the purpose of making noise, and simultaneously providing an additional muscle load for fitness purposes. The said combination weighted-percussive exercise device can be sewn into or onto the surface of a garment, inserted into and removed from a sealed compartment, or attached to a removable strap similar to a watch to be worn around various body parts as described in FIGS. 1 through 22A.

FIG. 24 illustrates a frontal view of an embodiment of the weighted-percussive exercise device, according to the present invention, whereby, rather than a hard exterior, the container of the individual percussive shaker instrument or the container of the combined weighted-percussive exercise device (labeled A), is a soft chamber, such as a sealable nylon bag or other desired material, and making use of Velcro, zipper, snaps or other common closure mechanism (labeled C), having located therein a plurality of hard pellets, balls or bells (labeled B) formed from material such as tungsten, plastic, steel, lead, or other noisemaking material, the quantity of which can amount to specifically desired weight intervals to provide an additional muscle load for fitness purposes, and simultaneously which are free to move about within the container for the purpose of making noise by hitting into each other when shaken. The said weighted percussive exercise device can be sewn into or onto the surface of a garment, inserted into and removed from a sealed compartment, or attached to a strap similar to a watch, to be worn around various body parts as described in FIGS. 1 through 22A.

FIGS. 25 A-C illustrate an embodiment of the weighted percussive exercise device, according to the present invention such that rather than housing the shaker instrument, weight, or the combined precisely weighted-percussive exercise device of the present invention (labeled FIG. 25 B) inside a designated compartment on a garment, sleeve band or belt, it is instead sewn onto the surface of the exercise garment, sleeve, band or belt, or contains a male or female mechanism of attachment such as Velcro, grommet, rivet, hook, or snap corresponding to a male or female mechanism of attachment on the surface of the exercise garment, sleeve, band or belt (labeled FIG. 25A), so that the shaker instrument, weight, or combined weighted-percussive device is thereby attached to the surface of the exercise garment, sleeve, band or belt (labeled FIG. 25 C).

FIGS. 25D-F illustrate an embodiment of the weighted percussive exercise device, according to the present invention such that rather than housing the shaker instrument, weight, and/or combined precisely weighted percussive exercise device inside a designated compartment on a garment, sleeve band or belt, it dangles from a garment such as trousers, shirt, shorts or other, to which it is attached using a common mechanism of attachment such as hooks, Velcro, snaps, zipper or sewn. Specifically in this illustration, FIG. 25 D depicts a frontal view of sports bra with a row of loops or “eyes” for corresponding hooks to attach around the entire length, front and back of the sport bra. FIG. 25E illustrates a typical Nigerian or Peruvian shaker comprised of a row of seeds or hooves (labeled A), which create a distinct shaker sound, and can be, but are not necessarily of a specified weight for the purpose of adding an additional muscle load for fitness, attached to a rope (labeled B) with companion hooks (labeled C) used to attach to the sports bra in FIG. 25D, which when united as in FIG. 25F create an embodiment of the weighted percussive exercise device, according to the present invention whereby the shaker, weight or combined percussive-weight dangles freely.

FIGS. 26A-D illustrate the electronic movement actuated means of producing sound or music embodiment. In FIG. 26A the electronic movement actuated means of producing sound or music (labeled A) is shown inside a compartment means (labeled B) such that as a user moves during exercise the electronic movement actuated means of producing sound or music moves inside the compartment means at the rhythm of the user's movements. The movement itself causes the electronic movement actuated means of producing sound or music to produce a user, instructor or physician selected audible sound such as that of a percussive instrument, music or some other sounds such as the sampling of the user's, or someone else's voice.

In FIG. 26B the electronic movement actuated means of producing sound or music (labeled A), similar to the device depicted in FIG. 26A creates a sound by movement, however in the embodiment depicted in FIG. 26B the sound produced by the electronic movement actuated means of producing sound or music is transmitted by a transmitter means (labeled B) to a receiver means (labeled C) and thereby rendered into audible sound by the speaker means (labeled D), which can be user worn device and earphones.

In FIG. 26C the electronic means of producing sound or music (labeled A), unlike the device depicted in FIG. 26A or FIG. 26B that create sound by movement, functions as a receiver means and thereby receives and renders into audible sound electronic signals from a transmitter source (labeled B). The embodiment in FIG. 26C can effectively be used in a group fitness setting where an instructor, and vicariously the class, is desirous of having the students become sound generating instruments in the class thereby augmenting the music being used in the class and especially its tempo. In this manner the sounds generated by each user in a class setting would have no risk of becoming a cacophony of off tempo sounds, but rather it would in essence be a living metronome motivating each student to keep up with the very sound that they are generating, in harmony with the instructor and the rest of the class.

It is also possible to combine all the features of the electronic means of producing sound or music such that they all may function at the same time, or individually or in any combination that a user may desire. In FIG. 26D the device is depicted with the direct sound producing means, transmitting means and receiving means as shown in FIGS. 26A-C all combined in one device (labeled A). In combination it is possible for instance to be transmitting, receiving and directly producing all different sounds or music, which in combination add to the tonal qualities of the experience and have the potential to permit a user to “feel” the music more directly and derive a greater sense of motivation in the exercise.

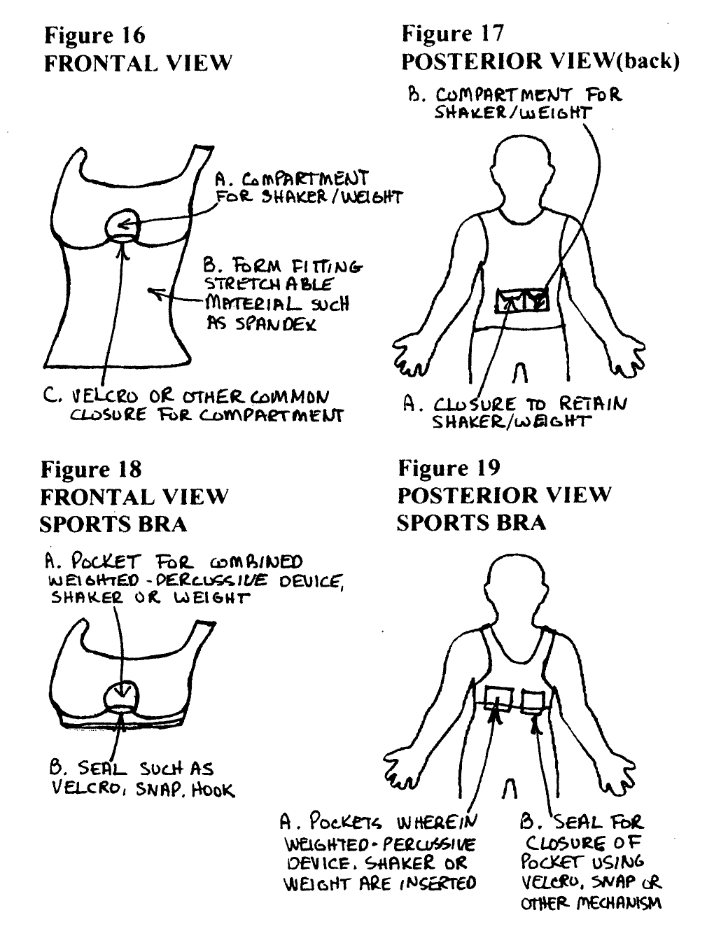

FIG. 27 depicts a user wearing the device as depicted in FIG. 16 which consists of a compartment for the shaker or movement (or electronically) actuated percussive musical device and weights (labeled A). The user is wearing beneath the garment and in contact with the skin a vital signs electronic monitoring means (labeled B). The electronic monitoring means may be placed on a user's body, external or internal, in any desired location where the electronic monitoring means is capable of detecting the user selected vital signs. The vital signs electronic monitoring means (labeled B) being in functional contact with the user's body such that it is capable of detecting, monitoring and transmitting the pulse, blood pressure, saturated blood oxygen level, body temperature, respiratory rate and data necessary to calculate the user's VO2 max to the vital signs read out and user exercise control programmer (labeled C). Traditionally heart rate monitors necessitated wearing a strap across the chest, however, newer version of heart rate and blood pressure monitors are available that simply strap to the wrist These modern blood pressure and heart rate wrist monitor devices can identify additional vital signs that may help an individual better assess his or her condition. This includes variability of the heart rate calorie burning and breathing rate. The vital signs read out and user exercise control programmer (labeled C) receives the data transmitted from the vital signs electronic monitoring means (labeled B) and by means of a microprocessor it processes the data and provides the user with a real time read out on the vital signs read out and user exercise control programmer (labeled C) of their vital signs. The user can also program the vital signs read out and user exercise control programmer (labeled C) to transmit a control signal to the shaker or electronic type movement actuated percussive musical device and weights (labeled A) and/or the pitchable music source (labeled D) to control either or both the volume of the user, instructor or physician selected music being played and/or the electronically produced percussive sound. The ideal embodiment of the present invention is an “all in one” such that the vitals monitor, the music source be it electronic or an actual instrument, and the electronic control panel are hosted in the same location on the body in one object, for example in a wristband. The electronic monitoring means in yet another embodiment can be incorporated into the shaker or electronic type movement actuated percussive musical device and weights.

In the most preferred embodiment the user would program the user exercise control programmer (labeled C) to a preset exercise program or a manual user, instructor or physician selected program. The user exercise control programmer (labeled C) could then be used to start the music playing remotely by wireless means on the pitchable music source (labeled D). The volume and speed (pitch) of the music would follow the selected exercise program to provide the pace and motivation to achieve the programmed fitness goal of the user. In selecting the exercise program the user, instructor or physician would also select a desired exertion level that would be determined by some vital sign parameter or preferably the percentage of VO2 max as calculated by the user exercise control programmer (labeled C) from the data it receives from the vital signs electronic monitoring means (labeled B). If the user exercise control programmer (labeled C) determines that the user is working below their selected level of exertion the user exercise control programmer (labeled C) will remotely incrementally increase the speed or pitch and the volume of the pitchable music source (labeled D) thereby causing the user to pick up their pace and in turn the exertion level will increase until the user reaches the desired level and that pitch and volume will remain until the program directs a differing exertion level be maintained. Vice versa, in the event the user exercise control programmer (labeled C) determines that the user is working above their selected level of exertion the user exercise control programmer (labeled C) will remotely incrementally decrease the speed or pitch and the volume of the pitchable music source (labeled D) thereby causing the user to slow down their pace and in turn the exertion level will decrease until the user reaches the desired level. If the vital signs electronic monitoring means (labeled B) detects vital signs that indicate the user is in physical danger by overexertion, elevated blood pressure beyond the user's normal range, rapid pulse and respiration rate, low blood oxygen saturation, etc., the user exercise control programmer (labeled C) can then turn off the music, wirelessly contact 911 if critical, or sound an alarm on the device or remotely, or any combination of these. In each embodiment the user, instructor or “physician could also manually adjust the parameters in any desired manner.

In another manner of use, the user exercise control panel notifies the user when the level of exertion has transitioned from one phase of workout into another: for example, when vital sign data confirm that the user has reached a pre-selected level of exertion which has been set at a given percentage of the maximal heart rate, and that level of achievement of the strenuous phase of aerobic exertion, the electronic control panel can notify the user by switching from one type of audible sound or instrumental sound, to another, so that the user does not have to read the display to know he has reached the new phase of his workout but rather can hear the change in instrument which signals to him his new level of exertion and phase of workout. The user exercise control panel can also inform him by a new audible signal when he has sustained the new phase for a pre-selected amount of time.

In the most preferred embodiment the user would wear the vital signs electronic monitoring device (labeled B) and the exercise control programmer (labeled C) continuously over a period of time sufficient for the user exercise control programmer (labeled C) to process and store the user's vital signs at work, play, during exercise and other activities and in sleep. The user exercise control programmer (labeled C) would process and store this information for purposes of monitoring fitness progress as these vitals are monitored in the future during exercise as well as during day to day activities. The user exercise control programmer (labeled C) by USB or other direct or wireless means could then be interfaced with a PC or personal electronic device for producing printouts and reports of their exercise progress and when needed to share this data with the user's doctors, etc.

A latitude of modification, change and substitution is intended in the foregoing disclosure, and in some instances some features of the invention will be employed without a corresponding use of other features; in other instances some features of the invention will be used in different combinations. Accordingly, it is appropriate that the appended claims be construed broadly and in a manner consistent with the spirit and scope of the invention herein to include various permutations, and to recognize that the briefly described drawings do not exhaust the possible permutations of the features of this invention which, for the purposes of enhanced and improved exercise, consistently seek to bring together, a specified weight with a percussive instrument, either each remaining a separate integral entity and simultaneously both being attached to a common garment; or each losing its individual nature, being instead integrated and combined into one precisely weighted percussive exercise device; and, in both cases to be worn in a hands-free manner on the body for the purpose of adding an audible rhythmic dimension to exercise while simultaneously increasing the muscle load on targeted areas of the body, resulting in an entirely new method of conducting an exercise program.

While the above descriptions of the invention, its parts, and operations contain many specificities, these should not be construed as limitations on the scope of the invention, but rather as exemplifications of present embodiments thereof. Many other variations are possible, for example, other embodiments, shapes, and sizes of the device can be constructed and designed to work by the principles of the present invention; various materials, colors and configurations can be employed in the device's design that would provide interesting embodiment differences to users. By way of example it would be obvious to one skilled in the art to select headphones, earphones, ear-buds, speakers, electronic sound wafers or the like (connected either by wireless or direct connection means) as the sound and/or music producing means of the current invention.

Accordingly, the scope of the invention should be determined not by the embodiments illustrated, but by the claims and their legal equivalents as filed herewith.

Having described my invention, I claim:

1. A weighted, musical, exercise device comprised of:
at least one user attachment means;
at least one compartment means attached to the user attachment means;
at least one sound producing means containable within the compartment means;
at least one vital signs detection means capable of detecting at least one the user's vital signs selected from the group consisting of a user's: pulse, resting heart rate, blood pressure, saturated blood oxygen level, body temperature, respiratory rate, or data necessary to calculate the user's VO2 max, percentage of maximal heart rate or other user selected measure of physical fitness;
at least one controller and processor means in electronic communication with the sound producing means and the vital signs detection means;
at least one music source means in electronic communication with the controller and processor means; and
at least one weight means of a known approximate weight containable within the compartment means.

2. A weighted, musical, exercise device comprised of:
at least one user attachment means;
at least one compartment means attached to the user attachment means;
at least one sound producing means containable within the compartment means, wherein the sound producing means is movement activated;
at least one vital signs detection means capable of detecting at least one the user's vital signs selected from the group consisting of user's: pulse, resting heart rate, blood pressure, saturated blood oxygen level, body temperature, respiratory rate, or data necessary to calculate the user's VO2 max, percentage of maximal heart rate or other user selected measure of physical fitness;
at least one controller and processor means in electronic communication with the sound producing means and the vital signs detection and vital signs recording means;
at least one vital signs recording means in electronic communication with the controller and processor means capable of recording over time at least one of the user's vital signs selected from the group consisting of a user's: pulse, resting heart rate blood pressure, saturated blood oxygen level, body temperature, respiratory rate, or data necessary to calculate changes over time in the user's VO2 max, percentage of maximal heart rate or other user selected measure of physical fitness;
at least one digital display means in electronic communication with the controller and processor means;
at least one music source means in electronic communication with the controller and processor means; and
at least one weight means of a known approximate weight contained within the compartment means.

3. The weighted, musical, exercise device of claim 1 wherein the user attachment means and the compartment means are comprised of:
elastomer fabric;
at least one attached adjustment strap; and
at least one securing means attached to the adjustment strap.

4. The weighted, musical, exercise device of claim 1 wherein the user attachment means is at least one article selected from the group consisting of: a sweatband adapted to be worn on a user's wrist, a sleeve adapted to be worn on a user's upper arm, a belt adapted to be worn on a user's abdomen, a sleeve adapted to be worn on a user's ankle, a sleeve adapted to be worn on a user's leg, a sleeve adapted to be worn on a user's hand, a sleeve adapted to be worn on a user's torso, or a sleeve adapted to be worn on a user's foot.

5. The weighted, musical, exercise device of claim 2 wherein the user attachment means is at least one article selected from the group consisting of: a sweatband adapted to be worn on a user's wrist, a sleeve adapted to be worn on a user's upper arm, a belt adapted to be worn on a user's abdomen, a sleeve adapted to be worn on a user's ankle, a sleeve adapted to be worn on a user's leg, a sleeve adapted to be worn on a user's hand, a sleeve adapted to be worn on a user's torso, or a sleeve adapted to be worn on a user's foot.

6. The weighted, musical, exercise device of claim 1 wherein the user attachment means is at least one article of clothing selected from the group consisting of: a shirt, a sweatshirt, a jacket, a bra, a pair of trousers, a pair of shorts, a pair of sweatpants, a pair of leggings, a headband, a sweatband, a belt, a hat or a shoe.

7. The weighted, musical, exercise device of claim 2 wherein the user attachment means is at least one article of clothing selected from the group consisting of: a shirt, a sweatshirt, a jacket, a bra, a pair of trousers, a pair of shorts, a pair of sweatpants, a pair of leggings, a headband, a sweatband, a belt, a hat or a shoe.

8. The weighted, musical, exercise device of claim 1 wherein the sound producing means is the weight means.

9. The weighted, musical, exercise device of claim 2 wherein the sound producing means is the weight means.

10. The weighted, musical, exercise device of claim 1 wherein the compartment means is a bag.

11. The weighted, musical, exercise device of claim 2 wherein the compartment means is a bag.

12. The weighted, musical, exercise device of claim 1 wherein the weight means and/or the sound producing means is attached to the user attachment means and not contained within the compartment means.

13. The weighted, musical, exercise device of claim 2 wherein the weight means and/or the sound producing means is attached to the user attachment means and not contained within the compartment means.

14. The weighted, musical, exercise device of claim 1 wherein the sound producing means is at least one sound producing device selected from the group consisting of an: agogo, agung/agong, agung a tamlang, ahoko, array mbira, babendil, bell, bianqing, bianzhong, bones, boomwhackers, cajón, carillon, castanets, caxixi, chime, ching, clapper, claves, cowbell, crotales, cymbal, zil (finger cymbal), cymbalum, electronic movement actuated means of producing sound or music, fangxiang, gamelan, american gamelan, gangsa, gendér, ugal, gandingan, gandingan a kayo, ganzá, ghatam, glass marimba, glass harmonica, glockenspiel, gong, guban, guiro, handbells, hang, hosho, ipu, kagul, kalimba, kulintang/kolintang, kulintang a kayo, kulintang a tiniok, lamellaphone, luntang, maracas, marimba, marimbaphone, mbira, metallophone, musical saw, rainstick, ranat ek lek, ranat thum lek, ratchet, rattle, riq, shekere, singing bowl, slit drum, spoons, steelpan, tambourine, triangle, trychel, timpani, tubular bells, udu, vibraphone, vibraslap, washboard, whip, xylophone, or xylorimba.

15. The weighted, musical, exercise device of claim 2 wherein the sound producing means is at least one sound producing device selected from the group consisting of an: agogo, agung/agong, agung a tamlang, ahoko, array mbira, babendil, bell, bianqing, bianzhong, bones, boomwhackers, cajón, carillon, castanets, caxixi, chime, ching, clapper, claves, cowbell, crotales, cymbal, zil (finger cymbal), cymbalum, electronic movement actuated means of producing sound or music, fangxiang, gamelan, american gamelan, gangsa, gendér, ugal, gandingan, gandingan a kayo, ganzá, ghatam, glass marimba, glass harmonica, glockenspiel, gong, guban, guiro, handbells, hang, hosho, ipu, kagul, kalimba, kulintang/kolintang, kulintang a kayo, kulintang a tiniok, lamellaphone, luntang, maracas, marimba, marimbaphone, mbira, metallophone, musical saw, rainstick, ranat ek lek, ranat thum lek, ratchet, rattle, riq, shekere, singing bowl, slit drum, spoons, steelpan, tambourine, triangle, trychel, timpani, tubular bells, udu, vibraphone, vibraslap, washboard, whip, xylophone, or xylorimba.

16. The weighted, musical, exercise device of claim 1 wherein the compartment means and the sound producing means combine to create a musical sound when the weighted, musical, exercise device is moved by a user.

17. The weighted, musical, exercise device of claim 2 wherein the compartment means and the sound producing means combine to create a musical sound when the weighted, musical, exercise device is moved by a user.

18. The weighted, musical, exercise device of claim 1 wherein the compartment means and the weight means combine to create a musical sound when the weighted, musical, exercise device is moved by a user.

19. The weighted, musical, exercise device of claim 2 wherein the compartment means and the weight means combine to create a musical sound when the weighted, musical, exercise device is moved by a user.

20. The weighted, musical, exercise device of claim 14 wherein the electronic movement actuated means of producing sound or music is at least one of the electronic devices selected from the group consisting of: at least one transmitter means whereby movement actuated sound is generated and transmitted to a user selected receiver to produce audible sound, at least one receiver means whereby audible sound can be generated from user selected transmission source, and at least one electronic movement actuated means of producing audible sound.

21. The weighted, musical, exercise device of claim 15 wherein the electronic movement actuated means of producing sound or music is at least one of the electronic devices selected from the group consisting of: at least one transmitter means whereby movement actuated sound is generated and transmitted to a user selected receiver to produce audible sound, at least one receiver means whereby audible sound can be generated from user selected transmission source, and at least one electronic movement actuated means of producing audible sound.

22. A method of using the weighted, musical, exercise device of claim 1 comprising the steps of:
selecting an exercise program;
programming the controller and processors means with the selected exercise program;
selecting music, a percussive rhythm or a metronome to accompany the selected exercise program;
attaching at least one of the weighted, musical, exercise devices of claim 1 by means of the user attachment means to a user's body by selecting at least one part of the user's body and attaching the weighted, musical, exercise devices of claim 1 to the selected parts of the user's body;
attaching the vital signs detection means of claim 1 to the user's body such that the user's selected vital signs can be detected by the vital signs detection means;
attaching the controller and processor means to the user's body such that the controller and processor means are accessible and visible to the user;
playing the selected music, rhythm or metronome at an audible volume;
exercising according to the selected exercise program; and
while exercising moving the selected parts of the user's body with the attached weighted, musical, exercise devices of claim 1 in rhythm to the selected music, rhythm or metronome thereby causing the sound producing means to produce an audible sound that has musical qualities when heard in rhythmic harmony with the selected music, rhythm or metronome, such that the audible sound is a real time indicator to the user of the phase of exercise the user has attained, and depending on the program setting as lead or follow, the type and tempo of audible musical sound will automatically change or change in response to monitored vital signs of the user as different thresholds of exercise expenditure are sought or attained, respectively, whereby the program of said device may lead the user to follow given rhythms in order to inspire exercise expenditure, or may follow user initiated movement to indicate to the user, by changes in instrument sound, the user's advancing or receding levels of exercise expenditure
after exercising storing information compiled by the exercise device of claim 1 for the purpose of monitoring fitness regimen progress and goals.

23. A method of using the weighted, musical, exercise device of claim 1 comprising the steps of:
selecting a rehabilitation program;
programming the controller and processors means with the selected rehabilitation program;
selecting music, a rhythm or a metronome to accompany the selected rehabilitation program;
attaching at least one of the weighted, musical, exercise devices of claim 1 by means of the user attachment means to a user's body by selecting at least one part of the user's body and attaching the weighted, musical, exercise devices of claim 1 to the selected parts of the user's body;
attaching the vital signs detection means of claim 1 to the user's body such that the user's selected vital signs can be detected by the vital signs detection means;
attaching the controller and processor means to the user's body such that the controller and processor means are accessible and visible to the user;
playing the selected music, rhythm or metronome at an audible volume;
rehabilitating according to the selected rehabilitation program;
while rehabilitating moving the selected parts of the user's body with the attached weighted, musical, exercise devices of claim 1 in rhythm to the selected music, rhythm or metronome thereby causing the sound producing means to produce an audible sound that has musical qualities when heard in rhythmic harmony with the selected music.

24. A method of using the weighted, musical, exercise device of claim 2 comprising the steps of:
selecting an exercise program;
programming the controller and processors means with the selected exercise program;
selecting music, a percussive rhythm or a metronome to accompany the selected exercise program;
attaching at least one of the weighted, musical, exercise devices of claim 2 by means of the user attachment means to a user's body by selecting at least one part of the user's body and attaching the weighted, musical, exercise devices of claim 2 to the selected parts of the user's body;
attaching the vital signs detection means of claim 2 to the user's body such that the user's selected vital signs can be detected by the vital signs detection means;
attaching the controller and processor means to the user's body such that the controller and processor means are accessible and visible to the user;
playing the selected music, rhythm or metronome at an audible volume;
exercising according to the selected exercise program; and
while exercising moving the selected parts of the user's body with the attached weighted, musical, exercise devices of claim 2 in rhythm to the selected music, rhythm or metronome thereby causing the sound producing means to produce an audible sound that has musical qualities when heard in rhythmic harmony with the selected music, rhythm or metronome, such that the audible sound is a real time indicator to the user of the phase of exercise the user has attained, and depending on the program setting as lead or follow, the type and tempo of audible musical sound will automatically change or change in response to monitored vital signs of the user as different thresholds of exercise expenditure are sought or attained, respectively, whereby the program of said device may lead the user to follow given rhythms in order to inspire exercise expenditure, or may follow user initiated movement to indicate to the user, by changes in instrument sound, the user's advancing or receding levels of exercise expenditure
after exercising storing information compiled by the exercise device of claim 2 for the purpose of monitoring fitness regimen progress and goals.

25. A method of using the weighted, musical, exercise device of claim 2 comprising the steps of:
selecting a rehabilitation program;
programming the controller and processors means with the selected rehabilitation program;
selecting music, a rhythm or a metronome to accompany the selected rehabilitation program;
attaching at least one of the weighted, musical, exercise devices of claim 2 by means of the user attachment means to a user's body by selecting at least one part of the user's body and attaching the weighted, musical, exercise devices of claim 2 to the selected parts of the user's body;
attaching the vital signs detection means of claim 2 to the user's body such that the user's selected vital signs can be detected by the vital signs detection means;
attaching the controller and processor means to the user's body such that the controller and processor means are accessible and visible to the user;
playing the selected music, rhythm or metronome at an audible volume;
rehabilitating according to the selected rehabilitation program;
while rehabilitating moving the selected parts of the user's body with the attached weighted, musical, exercise devices of claim 2 in rhythm to the selected music, rhythm or metronome thereby causing the sound producing means to produce an audible sound that has musical qualities when heard in rhythmic harmony with the selected music.
